(19) United States
(12) Patent Application Publication
(10) Pub. No.: US 2025/0161551 A1
(43) Pub. Date: May 22, 2025

(54) NEGATIVE PRESSURE WOUND THERAPY DRESSINGS, APPARATUSES, AND SYSTEMS

(71) Applicant: Solventum Intellectual Properties Company, Maplewood, MN (US)

(72) Inventors: Christopher BREACH, Bracknell (GB); Benjamin A. PRATT, Bracknell (GB)

(21) Appl. No.: 18/867,910

(22) PCT Filed: May 4, 2023
(86) PCT No.: PCT/IB2023/054648

§ 371 Date: Nov. 21, 2024

Related U.S. Application Data
(60) Provisional application No. 63/345,213, filed on May 24, 2022.

(51) Int. Cl. A61M 1/00; A61F 13/00; A61F 13/05

(52) U.S. Cl. CPC A61M 1/915; A61F 13/05; A61F 2013/00553

(57) ABSTRACT

Apparatuses, dressings, and systems for negative pressure wound therapy. The apparatus includes a first film layer, a second film layer, and a plurality of protrusions. The first film layer includes a first surface configured to contact the tissue site, a second surface opposite the first surface, and a plurality of fenestrations. The second film layer includes a first surface configured to couple to the second surface of the first film layer and a second surface opposite the first surface. The plurality of protrusions are disposed between the second surface of the first film layer and the first surface of the second film layer. The plurality of protrusions are integrally formed on at least one of the second surface of the first film layer or the first surface of the second film layer.

### CROSS-REFERENCE TO RELATED APPLICATIONS

This application is a U.S. National Stage Entry of International Application No. PCT/IB2023/054648, filed on May 4, 2023, which claims the benefit of priority to U.S. Provisional Application No. 63/345,213, filed on May 24, 2022, which is incorporated herein by reference in its entirety.

### TECHNICAL FIELD

The invention set forth in the appended claims relates generally to tissue treatment systems and more particularly, but without limitation, to simplified negative pressure wound therapy dressings, apparatuses, and systems capable of improved tissue compliance.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, dressings, and apparatuses for simplified therapy devices in a negative-pressure therapy environment are set forth in the appended claims. Illustrative embodiments are also provided to enable a person skilled in the art to make and use the claimed subject matter.

For example, in some embodiments, an apparatus for treating a tissue site with negative pressure is described. The apparatus can include a first film layer, a second film layer, and a plurality of protrusions. The first film layer can include a first surface configured to contact the tissue site, a second surface opposite the first surface, and a plurality of fenestrations. The second film layer can include a first surface configured to couple to the second surface of the first film layer and a second surface opposite the first surface. The plurality of protrusions can be disposed between the second surface of the first film layer and the first surface of the second film layer. The plurality of protrusions can be integrally formed on at least one of the second surface of the first film layer or the first surface of the second film layer.

In some example embodiments, the plurality of protrusions can be individually coupled to the second surface of the first film layer.

In some example embodiments, the plurality of protrusions can be embossed onto the second surface of the first film layer.

In some example embodiments, the plurality of protrusions can extend from the second surface of the first film layer towards the first surface of the second film layer. The plurality of fenestrations can be positioned between the plurality of protrusions.

In some example embodiments, the plurality of protrusions can be individually coupled to the first surface of the second film layer.

In some example embodiments, the plurality of protrusions can be embossed onto the first surface of the second film layer.

In some example embodiments, the plurality of protrusions can extend from the first surface of the second film layer towards the second surface of the first film layer. The plurality of fenestrations can be positioned between the plurality of protrusions.

In some example embodiments, the plurality of protrusions can be configured to manifold fluid between the first film layer and the second film layer.

In some example embodiments, one or more of the first surface of the first film layer, the second surface of the first film layer, or the first surface of the second film layer can include micro-patterning.

In some example embodiments, the plurality of protrusions can be solid cylindrical protrusions.

In some example embodiments, the plurality of protrusions can be hollow cylindrical protrusions.

In some example embodiments, the plurality of protrusions can be hollow castellated cylindrical protrusions.

In some example embodiments, the plurality of protrusions can be hollow flared cylindrical protrusions.

In some example embodiments, the plurality of protrusions can be solid truncated conical protrusions.

In some example embodiments, the plurality of protrusions can be hollow truncated conical protrusions.

Also described herein is a dressing for treating a tissue site with negative pressure. The dressing can include a cover, a tissue interface, and a sealing layer. The cover can be configured to create a seal at the tissue site. The tissue interface can be configured to be disposed between the cover and the tissue site. The tissue interface can include a first film layer, a second film layer, and a plurality of protrusions. The first film layer can include a first surface configured to contact the tissue site, a second surface opposite the first surface, and a plurality of fenestrations. The second film layer can include a first surface configured to couple to the second surface of the first film layer and a second surface opposite the first surface. The plurality of protrusions can be disposed between the second surface of the first film layer and the first surface of the second film layer. The plurality of protrusions can be integrally formed on at least one of the second surface of the first film layer or the first surface of the second film layer. The sealing layer can be configured to be disposed between the tissue interface and the tissue site.

In some example embodiments, the plurality of protrusions can be formed with a substrate material of at least one of the second surface of the first film layer or the first surface of the second film layer.

In some example embodiments, the plurality of protrusions can be formed from a substrate material of at least one of the second surface of the first film layer or the first surface of the second film layer.

Also described herein is a system for treating a tissue site. The system can include a dressing, a dressing interface, and a negative-pressure source. The dressing can include a cover, a tissue interface, and a sealing layer. The cover can be configured to create a seal at the tissue site. The tissue interface can be configured to be disposed between the cover and the tissue site. The tissue interface can include a first film layer, a second film layer, and a plurality of protrusions. The first film layer can include a first surface configured to contact the tissue site, a second surface opposite the first surface, and a plurality of fenestrations. The second film layer can include a first surface configured to couple to the second surface of the first film layer and a second surface opposite the first surface. The plurality of protrusions can be disposed between the second surface of the first film layer and the first surface of the second film layer. The plurality of protrusions can be integrally formed on at least one of the second surface of the first film layer or the first surface of the second film layer. The sealing layer can be configured to be disposed between the tissue interface and the tissue site. The dressing interface can be configured to couple to the dressing. The negative-pressure source can be configured to be fluidly coupled to the tissue interface through the dressing interface and an aperture in the second film layer.

In some example embodiments, the system can further include a support member configured to support the second film layer into sealing engagement with the dressing interface at the aperture in the second film layer. In some example embodiments, the support member can include a foam disk disposed between the second film layer and the first film layer. In some example embodiments, the support member can include a mounting ring configured to surround the aperture in the second film layer. In some example embodiments, the mounting ring can include a hydrocolloid ring positioned between the second surface of the second film layer and the dressing interface. In some example embodiments, the mounting ring can include a polymer ring with a higher stiffness than the second film layer.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is an exploded view of an example of the dressing of FIG. 1, illustrating additional details that may be associated with some example embodiments;

FIG. 3 is a side cross-sectional view of a portion of the dressing of FIG. 2, illustrating additional details that may be associated with some example embodiments;

FIG. 4 is a side cross-sectional view of a portion of the dressing of FIG. 1, illustrating additional details that may be associated with some example embodiments;

FIG. 5 is an isometric view of the dressing of FIG. 2, as assembled;

FIG. 6 is an isometric view of the dressing of FIG. 2, as assembled, and with an example dressing interface attached.

FIG. 7A is an isometric view of an example projection that may be included in an example of the dressing of FIG. 1;

FIG. 7B is another isometric view of an example projection that may be included in an example of the dressing of FIG. 1;

FIG. 7C is another isometric view of an example projection that may be included in an example of the dressing of FIG. 1;

FIG. 7D is another isometric view of an example projection that may be included in an example of the dressing of FIG. 1;

FIG. 7E is another isometric view of an example projection that may be included in an example of the dressing of FIG. 1;

FIG. 7F is another isometric view of an example projection that may be included in an example of the dressing of FIG. 1;

FIG. 8A is a cross-sectional view of the example dressing of FIG. 6, taken at line 8A-8A, applied to an example tissue site, and illustrating additional details associated with some examples of the therapy system of FIG. 1;

FIG. 8B is a detail view, taken at reference 8B in FIG. 8A, illustrating additional features, which may be associated with some examples of the dressing of FIG. 8A; and

FIG. 8C is a detail view illustrating additional features, which may be associated with the detail view of FIG. 8B in some implementations of the dressing of FIG. 8A.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

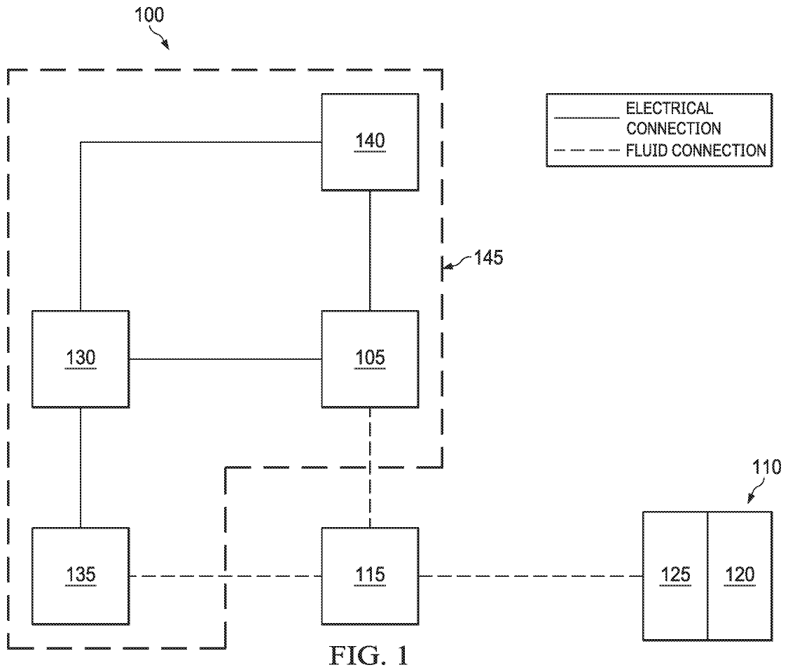

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from 3M Company.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit or a therapy device 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between-5 mm Hg (−667 Pa) and −500 mm Hg (−66.7 kPa). Common therapeutic ranges are between-50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, which may correspond to a pressure output of the negative-pressure source 105, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal. The first sensor 135 and the second sensor 140 are illustrated as optional features of some examples of the therapy device 145 in FIG. 1. However, in some examples, the first sensor 135 and/or the second sensor 140 can be omitted or moved to another portion of the therapy system 100, such as to a location external to the therapy device 145. In one such example, the second sensor 140 can be included as part of the therapy device 145 and the first sensor 135 can be configured as a pressure sensing device that can be fluidly coupled to the dressing 110 at multiple sensing locations external to the therapy device 145 as desired and as described herein.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across or through the tissue interface 120 under pressure. For example, a manifold may be adapted to receive negative pressure from a source and distribute negative pressure through multiple apertures across the tissue interface 120, which may have the effect of collecting fluid from a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from 3M Company.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from 3M Company. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and INSPIRER 2301 and INSPIRE® 2327 polyurethane films, commercially available from Exopack Advanced Coatings, Wrexham, United Kingdom. In some embodiments, the cover 125 may comprise INSPIRER 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the scaled therapeutic environment.

The process of reducing pressure may be described illustratively herein as “delivering,” “distributing,” or “generating” negative pressure, for example.

In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as limiting.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

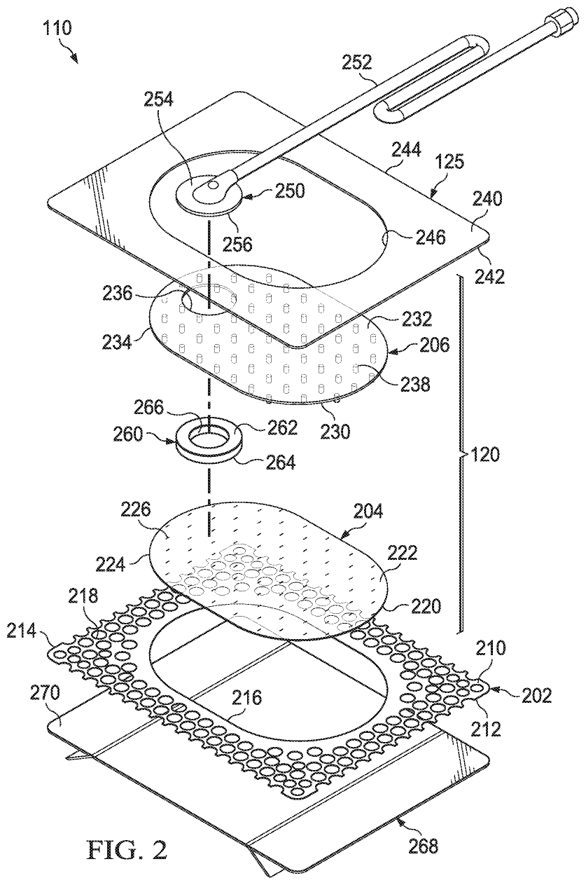

FIG. 2 is an exploded view of an example of the dressing 110 of FIG. 1, illustrating additional details that may be associated with some embodiments. In the example of FIG. 2, the dressing 110 may include a sealing layer 202, a first film layer 204, a second film layer 206, and the cover 125. In some examples, the first film layer 204 and the second film layer 206 may form the tissue interface 120 of the dressing 110. Further, the second film layer 206 may additionally or alternatively form a portion of the cover 125. The sealing layer 202 may be formed from a soft, pliable material suitable for providing a fluid seal with a tissue site, such as a suitable gel material, and may have a substantially flat surface. In various implementations, the scaling layer 202 may include, without limitation, a silicone gel, a soft silicone, hydrocolloid, hydrogel, polyurethane gel, polyolefin gel, hydrogenated styrenic copolymer gel, a foamed gel, soft closed-cell foams such as polyurethanes and polyolefins coated with adhesives, polyurethane, polyolefin, or hydrogenated styrenic copolymers. In various implementations, the sealing layer 202 may have a thickness in a range of about 200 micrometers to about 1,000 micrometers. In various implementations, the sealing layer 202 may be formed from hydrophobic or hydrophilic materials.

In various implementations, the sealing layer 202 may include or be formed from a hydrophobic or hydrophobic-coated material. For example, the sealing layer 202 may be formed by coating a spaced material, such as woven, nonwoven, molded, or extruded mesh, with a hydrophobic material such as a soft silicone.

The sealing layer 202 may have a top surface 210 opposite a bottom surface 212, a periphery 214 defined by an outer perimeter of the sealing layer 202, and a treatment aperture 216 formed through the sealing layer 202. The sealing layer 202 may also include a plurality of apertures 218 formed through the sealing layer 202. In various implementations, the plurality of apertures 218 may be formed through a region of the sealing layer 202 between the treatment aperture 216 and the periphery 214.

In various implementations, the apertures 218 may be formed by cutting, perforating, or applying local radio-frequency or ultrasonic energy through the sealing layer 202. In various implementations, the apertures 218 may be formed by other suitable techniques for forming an opening in the sealing layer 202. In various implementations, the apertures 218 may have a uniform distribution pattern, or may be randomly distributed. In various implementations, the apertures 218 may have many any combination of shapes, including circles, squares, stars, ovals, polygons, slits, complex curves, rectilinear shapes, or triangles.

In various implementations, each of the apertures 218 may have uniform or similar geometric properties. For example, each of the apertures 218 may be a circular aperture and have substantially the same diameter. In various implementations, each of the apertures 218 may have a diameter in a range of between about 1 millimeter and about 20 millimeters.

In various implementations, the geometric properties of the apertures 218 may vary. For example, the diameters of the apertures 218 may vary depending on the positioning of the respective apertures 218 in the sealing layer 202. In various implementations, at least some of the apertures 218 may have a diameter in a range of between about 5 millimeters to about 10 millimeters. In various implementations, at least some of the apertures 218 may have a diameter in a range of between about 7 millimeters and about 9 millimeters. In various implementations, the sealing layer 202 may include corners, and the apertures 218 disposed at or near the corners may have diameters in a range of between about 7 millimeters and about 8 millimeters.

In various implementations, at least some of the apertures 218 positioned near the periphery 214 may have an interior that is cut open or exposed at the periphery 214 and is in lateral communication in a lateral direction (relative to the top surface 210 and/or the bottom surface 212) with the periphery 214. In various implementations, the lateral direction may refer to a direction in a same plane as the top surface 210 and/or the bottom surface 212 and extending towards the periphery 214. In various implementations, at least some of the apertures 218 positioned proximate to or at the periphery 214 may be spaced substantially equidistantly around the periphery 214. Alternatively, in various implementations, the spacing of the apertures 218 proximate to or at the periphery 214 may be spaced irregularly.

The first film layer 204 may include a suitable structure for controlling or managing fluid flow. In various implementations, the first film layer 204 may be a fluid-control layer that includes a liquid-impermeable, vapor-permeable elastomeric material. In various implementations, the first film layer 204 may be formed from or include a polymer film. For example, in various implementations, the first film layer 204 may be formed from or include a polyolefin film, such as a polyethylene film. In various implementations, the first film layer 204 may be substantially clear or optically transparent. In various implementations, the first film layer 204 may be formed from or include the same material as the cover 125. In various implementations, the first film layer 204 may be formed from or include a biocompatible polyurethane film tested and certified according to the USP Class VI Standard. In various implementations, the first film layer 204 may also have a smooth or matte surface texture. In various implementations, the first film layer 204 may have a glossy or shiny finish equal to or exceeding a grade B3 according to the Society of Plastics Industry (SPI) standards. In various implementations, the surface of the first film layer 204 may be a substantially flat surface, with height variations in a range of about 0.2 millimeters to about 1 centimeter.

In various implementations, the first film layer 204 may be hydrophobic. The hydrophobicity of the first film layer 204 may vary but may have a contact angle with water of at least 90 degrees in some examples. In various implementations, the first film layer 204 may have a contact angle with water of no more than 150 degrees. In various implementations, the first film layer 204 may have a contact angle with water in a range of about 90 degrees to about 120 degrees, or in a range of about 120 degrees to about 150 degrees. Water contact angle may be measured using any standard apparatus. Although manual goniometers may be used to visually approximate contact angles, contact angle measuring instruments may often involve integrated systems that include a level stage, a liquid dropper (such as a syringe), a camera, and software designed to calculated contact angles more accurately and precisely. Non-limiting examples of such integrated systems include the FTÅ125, FTÅ200, FTÅ2000, and FTÅ4000 systems, all commercially available from First Ten Angstroms, Inc., of Portsmouth, Virginia, and the DTA25, DTA30, and DTA100 systems, all commercially available from Kruss GmbH of Hamburg, Germany. Unless otherwise specified, water contact angles herein are measured using deionized and/or distilled water on a level sample surface for a sessile drop added from a height of no more than five centimeters in air at 20-25° C. and 20-50% relative humidity. Contact angles herein represent averages of five to nine measured values, with the highest and lowest measured values discarded. In various implementations, the hydrophobicity of the first film layer 204 may be further enhanced with a hydrophobic coating of other materials, such as silicones and fluorocarbons.

The first film layer 204 may also be suitable for welding to other layers, including the second film layer 206. In various implementations, the first film layer 204 may be adapted for welding to polymers such as polyurethane, polyurethane films, and polyurethane foams using heat welding, radio-frequency (RF) welding, ultrasonic welding, or other methods. RF welding may be particularly suitable for more polar materials, such as polyurethane, polyamides, polyesters, and acrylates. Sacrificial polar interfaces may be used to facilitate RF welding of less polar film materials, such as polyethylene.

The area density of the first film layer 204 may vary according to a prescribed therapy or application. In various implementations, an area density of less than 40 grams per square meter may be suitable. In various implementations, the area density of the first film layer 204 may be in a range of about 20 grams per square meter to about 30 grams per square meter.

In various implementations, the first film layer 204 may be formed from or include a hydrophobic polymer, such as a polyethylene film. The simple and inert structure of polyethylene provides a surface that interacts little, if any, with biological tissues and fluids, and provides a surface that may encourage the free flow of liquids and exhibits a low adherence to tissues and fluids, properties that may be particularly advantageous for many applications. In various implementations, the first film layer 204 may be formed from other polymeric films such as polyurethanes, acrylics, polyolefins (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate styreneics, silicones, fluoropolymers, and acetates. In various implementations, the first film layer 204 may have a thickness in a range of about 20 micrometers to about 500 micrometers. In various implementations, the first film layer 204 may have a thickness of about 23 micrometers, about 25 micrometers, about 100 micrometers, about 250 micrometers, about 300 micrometers, and about 500 micrometers. In various implementations, the first film layer 204 may include a polar film suitable for lamination to the polyethylene film, such as polyamides, co-polyesters, ionomers, and acrylics. In various implementations, the first film layer 204 may include a tie layer to improve the bond between the polyethylene and polar film layers. In various implementations, the tie layer may include ethylene vinyl acetate or modified polyurethanes. In various implementations, the first film layer 204 may include an ethyl methyl acrylate (EMA) film.

As illustrated in FIG. 2, the first film layer 204 may have a first side or bottom surface 220 opposite a second side or top surface 222, and a periphery 224 defined by an outer perimeter of the first film layer 204. In some examples, the bottom surface 220 of the first film layer 204 may be a tissue contact surface of the tissue interface 120. In various implementations, the periphery 224 may be a stadium, disco rectangular, or obround shape. The first film layer 204 may also include one or more fluid passages 226 formed or disposed through the bottom surface 220 and the top surface 222 of the first film layer 204, and which may be distributed uniformly or randomly across the first film layer 204. In some embodiments, the first film layer 204 may optionally include micro-patterning (not shown) on either the bottom surface 220 or the top surface 222 of the first film layer 204. The micro-patterning may be a result of a manufacturing process to create the first film layer 204.

In various implementations, the fluid passages 226 may function as bi-directional and fluid-responsive valves. For example, each of the fluid passages 226 may be an elastic passage that is normally unstrained to prevent or substantially reduce fluid flow across the fluid passages 226 and can expand or open to allow fluid flow across the fluid passages 226 in response to a pressure gradient applied across the fluid passages 226. In various implementations, the fluid passages 226 may include perforations formed in the first film layer 204. Perforations may be formed by removing material from the first film layer 204 or cutting through the first film layer 204. In various implementations, cutting through the first film layer 204 may deform the edges of the perforations. In various implementations, the fluid passages 226 may be sufficiently narrow to form a seal or a fluid restriction to substantially reduce or prevent fluid flow across the fluid passages 226, particularly in the absence of a pressure differential. In various implementations, one or more of the fluid passages 226 may be an elastomeric valve that is normally closed when unstrained to prevent liquid flow across the valve, and that can open in response to a pressure gradient. In various implementations, the fluid passages 226 may be a plurality of fenestrations formed through the first film layer 204. Fenestrations may be formed by removing material from the first film layer 204, but the amount of material removed, and the resulting dimensions of the fenestrations may be up to an order of magnitude less than perforations and may not deform the edges.

In various implementations, the fluid passages 226 may include one or more slits, slots, or combinations of slits and slots in the first film layer 204. In various implementations, the fluid passages 226 may include linear slots having a length less than about five millimeters and a width less than about two millimeters. In various implementations, the length may be at least about two millimeters, and the width may be at least about 0.5 millimeters. In various implementations, the length may be in a range of about two millimeters to about five millimeters and the width may be in a range of about 0.5 millimeters to about two millimeters, with a tolerance of about 0.1 millimeters. In various implementations, the length may be about three millimeters. Such dimensions and tolerances may be achieved with a laser cutter, for example. In various implementations, slots of such configurations may function as imperfect valves that substantially reduce liquid flow in a normally closed or resting state. Such slots may form a flow restriction without being completely closed or sealed. The slots can expand or open wider in response to a pressure gradient applied across the slot to allow increased liquid flow through the slot.

In various implementations, the fluid passages 226 may include linear slits having a length of less than about five millimeters. In various implementations, the length of the linear slits may be at least about two millimeters. In various implementations, the length of the linear slits may be in a range of about two millimeters to about five millimeters, with a tolerance of about 0.1 millimeters. In various implementations, the length of the linear slits may be about three millimeters.

The second film layer 206 may have a first side or bottom surface 230 opposite a second side or top surface 232, and a periphery 234 defined by a perimeter of the second film layer 206. The bottom surface 230 of the second film layer 206 may be disposed adjacent to the top surface 222 of the first film layer 204. A negative-pressure aperture, such as aperture 236, may be formed through the second film layer 206. In various implementations, the second film layer 206 may be formed from or include any of the materials previously described with respect to the cover 125 and/or the first film layer 204. In some embodiments, the second film layer 206 may optionally include micro-patterning (not shown) on either the bottom surface 230 or the top surface 232 of the second film layer 206. The micro-patterning may be a result of a manufacturing process to create the second film layer 206.

In various implementations, projections such as a plurality of protrusions 238 may be formed on the bottom surface 230 of the second film layer 206. In various implementations, the plurality of protrusions 238 may form a grid pattern. For example, the plurality of protrusions 238 may be arranged in a pattern of rows and columns such that the center of each protrusion 238 may be aligned with the center of each other protrusion 238 within a row, and the center of each protrusions 238 may be aligned with the center of each other protrusions 238 within a column. In other implementations, the plurality of protrusions 238 may be arranged in a different pattern or may be arranged randomly. In various implementations, each of the plurality of protrusions 238 may be substantially circular in profile and protrude outwardly in a substantially orthogonal manner from the plane of the bottom surface 230 of the second film layer 206. In various implementations, each protrusion 238 may have a diameter of about three millimeters and a height of in a range of about 0.5 millimeters to about 3 millimeters. In various implementations, each protrusion 238 may have a height of about 2.5 millimeters. In various implementations, each protrusion 238 may have a height of about 3 millimeters. In various implementations, each protrusion 238 within a row may be spaced a distance of about four millimeters on center from an adjacent protrusion 238 within a row, and each protrusion 238 within a column may be spaced a distance of about four millimeters on center from an adjacent protrusion 238 within a column. In various implementations, the plurality of protrusions 238 may be right cylinders with hemispherical ends, such as half-capsules, and may be formed on and protrude substantially away from the bottom surface 230 of the second film layer 206 in a direction substantially normal to the bottom surface 230. In various implementations, each of the plurality of protrusions 238 may have a height in a range of about 2.5 millimeters to about three millimeters.

As shown in FIG. 2, examples of the dressing 110 may include the cover 125 having a top surface 240 opposite a bottom surface 242, and a periphery 244 defined by an outer perimeter of the cover 125. A central aperture 246 may be formed through the cover 125. In some examples, the second film layer 206 may form a portion of the cover 125 by being coupled to the cover 125 and exposed through the central aperture 246, or the second film layer 206 may be integrally formed with the cover 125 as a central portion of the cover 125.

In various implementations, the periphery 214 of the sealing layer 202 may be substantially coextensive with the periphery 244 of the cover 125. In various implementations, the periphery 224 of the first film layer 204 and the periphery 234 of the second film layer 206 may be substantially coextensive. In various implementations, the outline of the treatment aperture 216 of the sealing layer 202 may be substantially coextensive with the outline of the central aperture 246 of the cover 125. In various implementations, the outlines of the treatment aperture 216 and the central aperture 246 may be substantially similar to the outlines of the periphery 224 and the periphery 234. In various implementations, the outlines of the treatment aperture 216 and the central aperture 246 may be substantially similar to but scaled down from the outlines of the periphery 224 and the periphery 234. In assembled form, the sealing layer 202, the first film layer 204, the second film layer 206, and the cover 125 may be stacked such that the periphery 214 is aligned with the periphery 244, and the periphery 224 is aligned with the periphery 234. In various implementations, the treatment aperture 216 may be aligned with the central aperture 246, and the periphery 224 and the periphery 234 are positioned such that they are aligned with and evenly extend past or overlap the outlines of the treatment aperture 216 and the central aperture 246.

In various implementations, a portion of the top surface 210 of the sealing layer 202 around the treatment aperture 216 may be coupled to a portion of the bottom surface 220 of the first film layer 204 near the periphery 224, and a portion of the bottom surface 242 of the cover 125 around the central aperture 246 may be coupled to a portion of the top surface 232 of the second film layer 206 near the periphery 234. In various implementations, a portion of the top surface 210 of the sealing layer 202 between the periphery 214 and the treatment aperture 216 may be coupled to a portion of the bottom surface 242 of the cover 125 between the periphery 244 and the central aperture 246.

Some examples of the dressing 110 also include a dressing interface 250 and a fluid conductor 252. In various implementations, the fluid conductor 252 may be a flexible tube that can be fluidly coupled on one end to the dressing interface 250. In various implementations, the dressing interface 250 may be an elbow connector that can be placed over the aperture 236 to provide a fluid path between the fluid conductor 252 and the interior of the dressing 110. For example, the dressing interface 250 may be coupled to the top surface 232 of the second film layer 206 over the aperture 236. In some examples, the second film layer 206 may be formed integrally with the cover 125 as a unified or single layer of material such that the second film layer 206 forms a central portion of the cover 125. Further, in some examples, the second film layer 206 may be referred to herein as a cover regardless of whether the second film layer 206 is formed integrally with the cover 125. In some examples, the dressing interface 250 may include a housing 254, and the housing 254 may include a mounting surface 256 configured to be coupled to a cover or a first portion of the dressing 110, such as the second film layer 206, around the aperture 236.

Further, some examples of the dressing 110 may optionally include a support member 260 configured to be coupled between the second film layer 206 and the first film layer 204. The support member 260 may include a first end 262 in fluid communication with a second end 264 through the support member 260. The first end 262 of the support member 260 is configured to be fluidly coupled to the bottom surface 230 of the second film layer 206 and the second end 264 of the support member 260 is configured to be positioned proximate to the top surface 222 of the first film layer 204. The support member 260 may include a central aperture 266 in some embodiments. A portion of the first end 262 of the support member 260 may couple with the mounting surface 256 of the dressing interface 250 through the second film layer 206. The support member 260 may be configured to support the second film layer 206 into sealing engagement with the dressing interface 250 at the aperture 236 of the second film layer 206. In some embodiments, the support member 260 may be a foam disk or a mounting ring that may be configured to surround the aperture 236 of the second film layer 206. In some examples, the support member 260 can be disposed between the second film layer 206 and the first film layer 204, as shown in FIG. 2, to press, support, or otherwise add rigidity to a portion of the second film layer 206 around the aperture 236 that can be coupled to the mounting surface 256. In such an example, a portion of the second film layer 206 around the aperture 236 can be captured or positioned between the mounting surface 256 and the first end 262 of the support member 260. In some embodiments where the support member 260 is a mounting ring, the mounting ring may be or may include a polymer or a gel, such as a hydrocolloid, that may be positioned between the top surface 232 of the second film layer 206 and the dressing interface 250. The mounting ring may include a higher stiffness or rigidity than the second film layer 206.

As illustrated in FIG. 2, the dressing interface 250 and the support member 260 are substantially circular. In some embodiments, the dressing interface 250 and/or the support member 260 may be rectangular, ovular, polygonal, or another shape that may provide a sufficient seal between the dressing interface 250 and the dressing 110 to ensure the delivery of negative pressure to a tissue site through the tissue interface 120.

As illustrated in FIG. 2, some examples of the dressing 110 may include a release liner 268 to protect the sealing layer 202 and the adhesive coated on the bottom surface 242 of the cover 125 prior to use. The release liner 268 may also provide stiffness to assist with, for example, deployment of the dressing 110. In various implementations, the release liner may include a polyethylene terephthalate (PET) or similar polar semi-crystalline polymer. The use of a polar semi-crystalline polymer for the release liner 268 may substantially preclude wrinkling or other deformation of the dressing 110. The polar semi-crystalline polymer may be highly orientated and resistant to softening, swelling, or other deformation that may occur when objects are brought into contact with the layers and/or components of the dressing 110, or when the dressing 110 is subjected to temperature or environmental variations, or during sterilization. Further, a release agent may be disposed on a top surface 270 of the release liner 268 that is configured to contact the bottom surface 212 of the sealing layer 202 and the adhesive disposed on the bottom surface 242 of the cover 125. For example, the release agent may be a silicone coating and may have a release factor suitable to facilitate removal of the release liner 268 by hand and without damaging or deforming the dressing 110. In various implementations, the release agent may be a fluorocarbon or a fluorosilicone. In various implementations, the release liner 268 may be uncoated or otherwise used without a release agent.

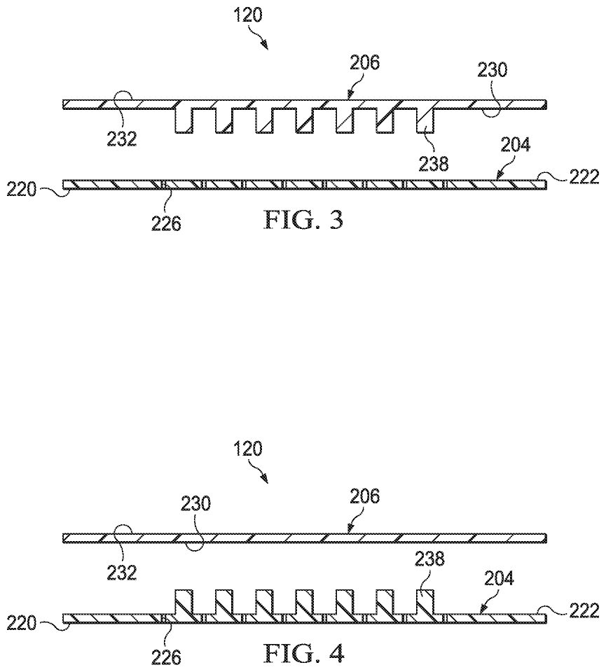

FIG. 3 is an exploded side cut-away view of the tissue interface 120 of FIG. 2. The tissue interface 120 may include the first film layer 204 and the second film layer 206 and the plurality of protrusions 238 may be disposed between the bottom surface 230 of the second film layer 206 and the top surface 222 of the first film layer 204 as described. However, as shown in FIG. 3, in some examples, the plurality of protrusions 238 may extend from the bottom surface 230 of the second film layer 206 towards the top surface 222 of the first film layer 204. The plurality of protrusions 238 may be integrally formed on the bottom surface 230 of the second film layer 206. In some embodiments, the plurality of protrusions 238 may be individually coupled to the bottom surface 230 of the second film layer 206. In some embodiments, the plurality of protrusions 238 may be embossed onto the bottom surface 230 of the second film layer 206. The plurality of protrusions 238 may be positioned on the bottom surface 230 of the second film layer 206 such that the plurality of protrusions 238 are positioned between the one or more fluid passages 226 of the first film layer 204. In some embodiments, the plurality of protrusions 238 may be formed as a unitary component with a substrate material of the bottom surface 230 of the second film layer 206. Additionally or alternatively, the plurality of protrusions 238 may be formed from the same substrate material as the bottom surface 230 of the second film layer 206.

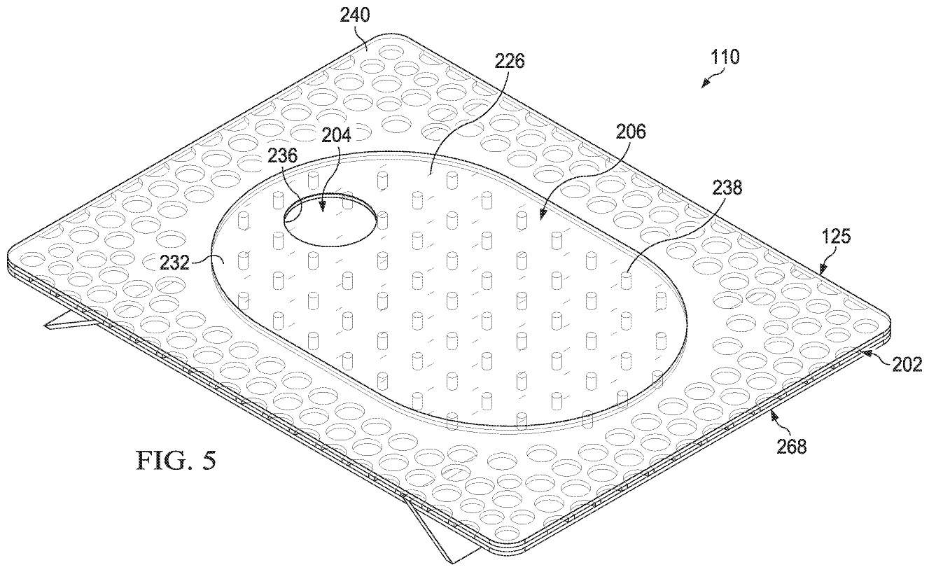

FIG. 4 is an exploded side cut-away view of another embodiment of the tissue interface 120 of the dressing 110. The tissue interface 120 may include the first film layer 204 and the second film layer 206 and the plurality of protrusions 238 may be disposed between the bottom surface 230 of the second film layer 206 and the top surface 222 of the first film layer 204. However, as shown in FIG. 4, in some examples, the plurality of protrusions 238 may extend from the top surface 222 of the first film layer 204 towards the bottom surface 230 of the second film layer 206. The plurality of protrusions 238 may be integrally formed on the top surface 222 of the first film layer 204. In some embodiments, the plurality of protrusions 238 may be individually coupled to the top surface 222 of the first film layer 204. In some embodiments, the plurality of protrusions 238 may be embossed onto the top surface 222 of the first film layer 204. The plurality of protrusions 238 may be positioned on the top surface 222 of the first film layer 204 such that the plurality of protrusions 238 are positioned between the one or more fluid passages 226 of the first film layer 204. In some embodiments, the plurality of protrusions 238 may be formed as a unitary component with a substrate material of the top surface 222 of the first film layer 204. Additionally or alternatively, the plurality of protrusions 238 may be formed from the same substrate material as the top surface 222 of the first film layer 204.

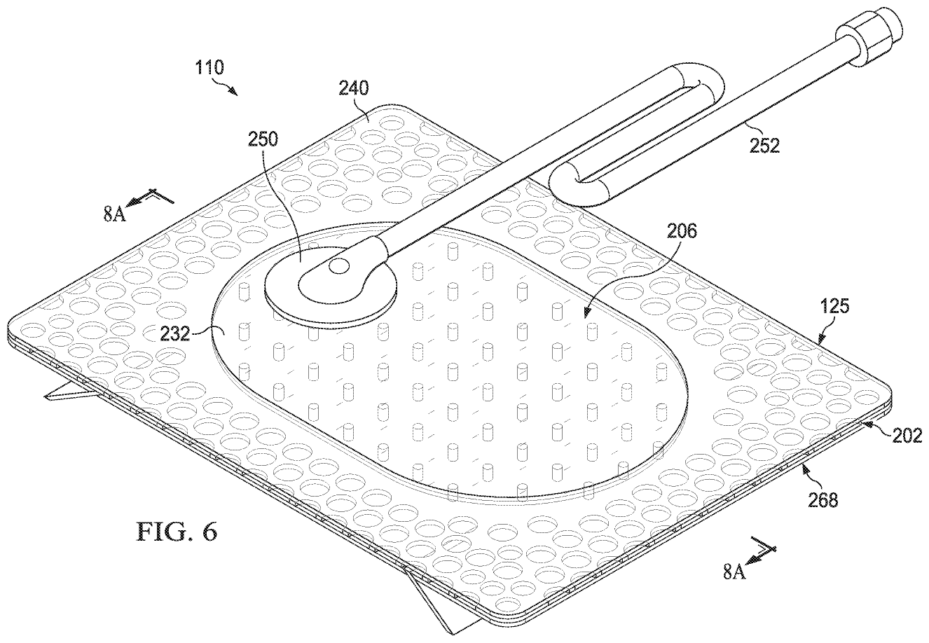

FIG. 5 is an isometric view of an assembled example of the dressing 110 of FIG. 2. As shown in FIG. 5, in some examples, the cover 125, the second film layer 206, and/or the first film layer 204 may be substantially clear or optically transparent, allowing for visualization of a tissue site through the layers of the dressing 110.

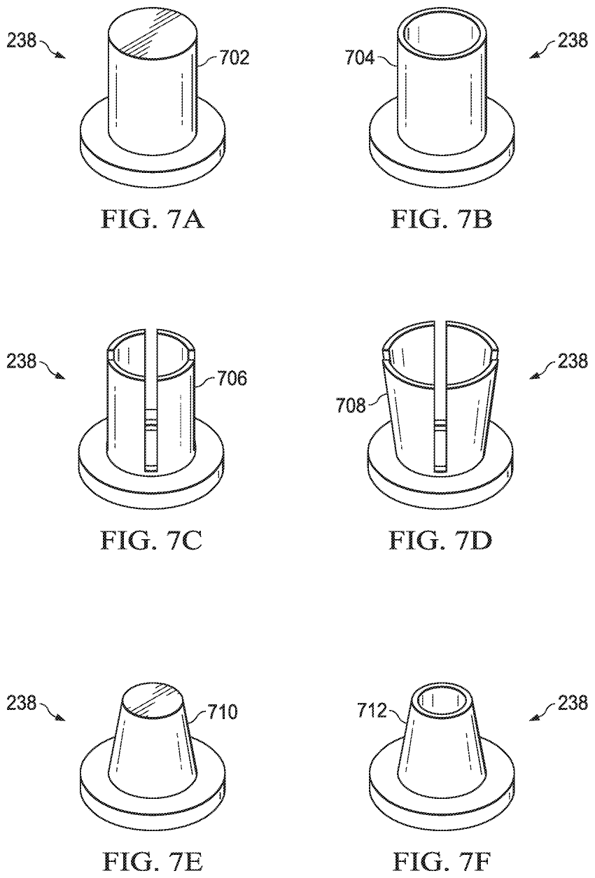

FIG. 6 is an isometric view of the assembled dressing 110 of FIGS. 2 and 5 with the dressing interface 250 attached. As shown in FIG. 6 with reference to FIG. 5, in some examples, the dressing interface 250 and the fluid conductor 252 may be attached to the top surface 232 and/or the top surface 240 and fluidly coupled to the tissue interface 120 within the interior of the dressing 110 via the aperture 236. In some embodiments, the support member 260 may be disposed between the first film layer 204 and the second film layer 206 to provide support to connect the dressing interface 250 to the dressing 110. In various implementations, the fluid conductor 252 may be connected to the negative-pressure source 105.

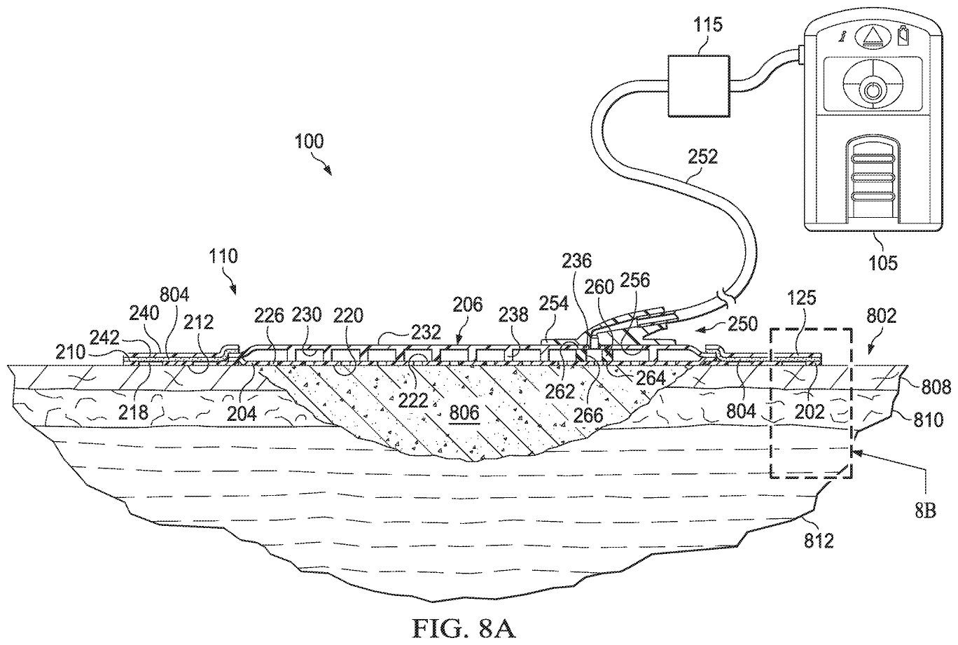

Referring collectively to FIGS. 7A to 7F, different embodiments of the plurality of protrusions 238 are shown. As shown in FIG. 7A, the plurality of protrusions 238 may be solid cylindrical protrusions 702. As shown in FIG. 7B, the plurality of protrusions 238 may be hollow cylindrical protrusions 704. As shown in FIG. 7C, the plurality of protrusions 238 may be castellated hollow cylindrical protrusions 706. As shown in FIG. 7D, the plurality of protrusions 238 may be castellated hollow flared cylindrical protrusions 708. In some embodiments, the hollow flared cylindrical protrusions may not be castellated. As shown in FIG. 7E, the plurality of protrusions 238 may be solid truncated conical protrusions 710. As shown in FIG. 7F, the plurality of protrusions 238 may be hollow truncated conical protrusions 712. Each of the embodiments of the plurality of protrusions 238 shown in FIGS. 7A to 7F may create pathways between the first film layer 204 and the second film layer 206 of the dressing 110. In other embodiments, the plurality of protrusions 238 may be other shapes or sizes that may create pathways between the first film layer 204 and the second film layer 206. Further, any of the embodiments of the plurality of projections of FIGS. 7A to 7F may be integrally formed with the bottom surface 230 of the second film layer 206 or with the top surface 222 of the first film layer 204.

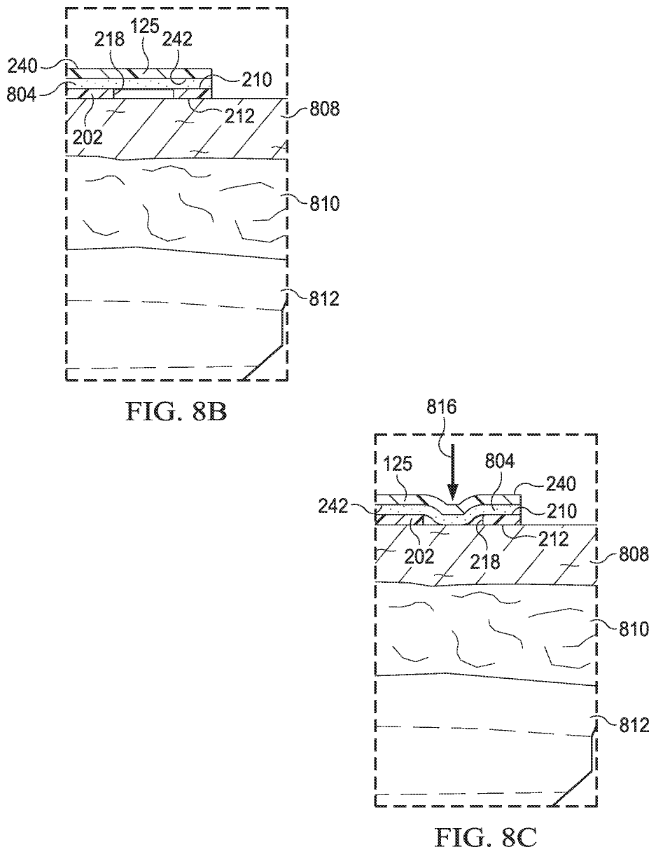

FIG. 8A is a cross-sectional view of the dressing 110 of FIG. 6, taken at line 8A-8A, applied to a tissue site 802, and illustrating additional details associated with the therapy system 100 of FIG. 1. As shown in FIG. 8A, in some examples, the bottom surface 220 of the first film layer 204 may be configured to be positioned in direct contact with a tissue site.

Further, as shown in FIG. 8A, in various implementations, the bottom surface 242 of the cover 125 may be coated with an adhesive layer 804, and at least a portion of the cover 125 may be coupled to at least a portion of the top surface 210 of the sealing layer 202 with the adhesive layer 804. The adhesive layer 804 may be any of the attachment devices previously discussed with reference to FIG. 1. At least a portion of the bottom surface 242 of the cover 125 may be coupled to at least a portion of the top surface 232 of the second film layer 206, for example, at the periphery 234, by the adhesive layer 804. In various implementations, at least a portion of the bottom surface 220 of the first film layer 204, for example, at the periphery 224, may be coupled to at least a portion of the top surface 210 of the sealing layer 202.

In some examples, the dressing 110 may be applied to the tissue site 802 and cover a wound 806. The tissue site 802 may be or may include a defect or targeted treatment site, such as the wound 806, which may be partially or completely filled or covered by the dressing 110. In some examples, the wound 806 may be in epidermis 808. In some examples, the wound 806 may extend through the epidermis 808 and into a dermis 810. In some examples, as shown in FIG. 8A, the wound 806 may extend through the epidermis 808 and the dermis 810 into a subcutaneous tissue 812. In some applications, at least a portion of the bottom surface 212 of the sealing layer 202 may be brought into contact with a portion of the epidermis 808 surrounding the wound 806, and at least a portion of the bottom surface 220 of the first film layer 204 may be placed within, over, on, against, or otherwise proximate to the wound 806.

In operation, negative pressure may be provided to the wound 806, and/or fluid may be removed from the wound 806 by the negative-pressure source 105. For example, fluid may travel from the wound 806 through the fluid passages 226 of the first film layer 204 and through the aperture 236 of the second film layer 206 and into the dressing interface 250. From the dressing interface 250, the fluid may flow towards to the negative-pressure source 105 through the fluid conductor 252 and/or the container 115.

FIG. 8B is a detail view, taken at reference 8B in FIG. 8A, illustrating details that may be associated with some example embodiments of the dressing 110 of FIG. 8A. In various implementations, the sealing layer 202 may be sufficiently tacky at the bottom surface 212 to hold the dressing 110 in position relative to the epidermis 808 and/or the wound 806, while allowing the dressing 110 to be removed or repositioned without trauma to the tissue site 802. In various implementations, the sealing layer 202 may be formed of a silicone material, which may form sealing couplings at the bottom surface 212 with the epidermis 808. In various implementations, the bond strength or tackiness of the sealing couplings may have a peel adhesion or resistance to being peeled from a stainless steel material between about 0.5 N/25 mm to about 1.5 N/25 mm on a stainless steel substrate at about 25° C. at about 50% relative humidity based on ASTM D3330. The sealing layer 202 may achieve this bond strength after a contact time of less than about 60 seconds. Tackiness may be considered a bond strength of an adhesive after a very low contact time between the adhesive and a substrate. In various implementations, the sealing layer 202 may have a thickness in a range of about 200 micrometers to about 1,000 micrometers. Removing the release liner 268 may also expose the adhesive layer 804 through the apertures 218 of the sealing layer 202. In the assembled state, the thickness of the sealing layer 202 may create a gap between the adhesive layer 804 and the epidermis 808 through the apertures 218 of the scaling layer 202 such that the adhesive layer 804 is not in contact with the epidermis 808.

FIG. 8C is a detail view illustrating additional details that may be associated with the detail view of FIG. 8B in some implementations of the dressing 110 of FIG. 8A. FIG. 8C illustrates the adhesive layer 804 after a portion of it has been brought into contact with the epidermis 808 by a force vector 816 applied to the top surface 240 of the cover 125 at the apertures 218. In use, if the assembled dressing 110 is at the desired location, the force vector 816 may be applied to the top surface 240 at the apertures 218 to cause at least a portion of the adhesive layer 804 to be pressed at least partially into contact with the epidermis 808 through at least one or more of the apertures 218 to form bonding couplings. The bonding couplings may provide secure, releasable mechanical fixation of the dressing 110 to the epidermis 808. In various implementations, the sealing couplings may not be as mechanically strong as the bonding couplings. The bonding couplings may anchor the dressing 110 to the epidermis 808, inhibiting and/or substantially preventing migration of the dressing 110.

The systems, dressings, and apparatuses described herein may provide significant advantages. For example, the configuration of the dressing 110 may provide for a thin overall construction with a minimal number of dressing layers. As such, the dressing 110 may be configured to be more compliant or conformable to a particular tissue site. For example, the dressing 110 may be configured to fit into small, deep, or irregularly shaped wounds or tissue sites. Referring back to FIG. 8A, the compliant configuration of the dressing 110 can permit the bottom surface 220 of the first film layer 204 to be pressed into or drawn into contact with a surface of the wound 806, which may extend through portions of the epidermis 808, dermis 810, and subcutaneous tissue 812 as illustrated. The dressing 110 may be pressed into contact with the wound 806 by a user or by operation of the application of negative pressure to the tissue site, for example. Further, the dressing 110 is a simplified design of the tissue interface 120 that includes two layers, the first film layer 204 and the second film layer 206. This two-layer design may simplify manufacturing and assembly of the tissue interface 120 and/or the dressing 110 by providing a simplified structure and reduced material usage. Further, the dressing 110 may have improved optical clarity when the dressing 110 is transparent because of the two-layer design of the tissue interface 120.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for treating a tissue site with negative pressure, the apparatus comprising:
a first film layer having a first surface configured to contact the tissue site, a second surface opposite the first surface, and a plurality of fenestrations;
a second film layer having a first surface configured to couple to the second surface of the first film layer, and a second surface opposite the first surface; and
a plurality of protrusions disposed between the second surface of the first film layer and the first surface of the second film layer, the plurality of protrusions integrally formed on at least one of the second surface of the first film layer or the first surface of the second film layer.

2. The apparatus of claim 1, wherein the plurality of protrusions are individually coupled to the second surface of the first film layer.

3. The apparatus of claim 1, wherein the plurality of protrusions are embossed onto the second surface of the first film layer.

4. The apparatus of claim 1, wherein the plurality of protrusions extend from the second surface of the first film layer towards the first surface of the second film layer, and wherein the plurality of fenestrations are positioned between the plurality of protrusions.

5. The apparatus of claim 1, wherein the plurality of protrusions are individually coupled to the first surface of the second film layer.

6. The apparatus of claim 1, wherein the plurality of protrusions are embossed onto the first surface of the second film layer.

7. The apparatus of claim 1, wherein the plurality of protrusions extend from the first surface of the second film layer towards the second surface of the first film layer, and wherein the plurality of fenestrations are positioned between the plurality of protrusions.

8. The apparatus of claim 1, wherein the plurality of protrusions are configured to manifold fluid between the first film layer and the second film layer.

9. The apparatus of claim 1, wherein one or more of the first surface of the first film layer, the second surface of the first film layer, or the first surface of the second film layer comprises micro-patterning.

10. The apparatus of claim 1, wherein one or more of the plurality of protrusions are solid cylindrical protrusions or solid truncated conical protrusions.

11. The apparatus of claim 1, wherein the plurality of protrusions are hollow.

12. The apparatus of claim 11, wherein one or more of the plurality of protrusions further comprise a cylindrical, castellated cylindrical, flared cylindrical, or truncated conical shape.

13. (canceled)

14. (canceled)

15. (canceled)

16. A dressing for treating a tissue site with negative pressure, the dressing comprising:
a cover configured to create a seal at the tissue site;
a tissue interface configured to be disposed between the cover and the tissue site, the tissue interface comprising:
a first film layer having a first surface configured to contact the tissue site, a second surface opposite the first surface, and a plurality of fenestrations;
a second film layer having a first surface configured to couple to the second surface of the first film layer, and a second surface opposite the first surface; and
a plurality of protrusions disposed between the second surface of the first film layer and the first surface of the second film layer, the plurality of protrusions integrally formed on at least one of the second surface of the first film layer or the first surface of the second film layer; and
a sealing layer configured to be disposed between the tissue interface and the tissue site.

17. The dressing of claim 16, wherein the plurality of protrusions are formed with a substrate material of at least one of the second surface of the first film layer or the first surface of the second film layer.

18. (canceled)

19. A system for treating a tissue site, the system comprising:
the dressing of claim 16;
a dressing interface configured to couple to the dressing; and
a negative-pressure source configured to be fluidly coupled to the tissue interface through the dressing interface and an aperture in the second film layer.

20. The system of claim 19, further comprising a support member configured to support the second film layer into sealing engagement with the dressing interface at the aperture in the second film layer.

21. The system of claim 20, wherein the support member comprises a foam disk disposed between the second film layer and the first film layer.

22. The system of claim 20, wherein the support member comprises a mounting ring configured to surround the aperture in the second film layer.

23. The system of claim 22, wherein the mounting ring comprises a hydrocolloid ring positioned between the second surface of the second film layer and the dressing interface.

24. The system of claim 22, wherein the mounting ring comprises a polymer ring including a higher stiffness than the second film layer.

25. (canceled)
